(19) United States
(12) Patent Application Publication
(10) Pub. No.: US 2026/0335088 A1
(43) Pub. Date: Oct. 29, 2026

(54) BIODEGRADABLE IMPLANT

(71) Applicant: BELLASENO GMBH, Leipzig (DE)

(72) Inventors: Mohit Prashant Chhaya, Leipzig (DE); Navid Khani, Leipzig (DE); Tobias Grossner, Heidelberg (DE); Alexander Schneider, Leipzig (DE); Laura Salucci, Leipzig (DE)

(21) Appl. No.: 19/099,174

(22) PCT Filed: Jul. 8, 2023
(86) PCT No.: PCT/EP2023/071875

§ 371 Date: Jan. 28, 2025

(30) Foreign Application Priority Data
Aug. 5, 2022 (EP) 22189015.5

(51) Int. Cl. A61F 2/28

(52) U.S. Cl. CPC A61F 2/2846; A61F 2002/2825

(57) ABSTRACT

In various embodiments an implant (20, 70) for containing a substance, such as a biological cell material and/or synthetic bone graft material, is provided, comprising a mesh (10) formed into a continuous sidewall; wherein the mesh (10) comprises a plurality of circumferential line segments (11), which are arranged above one another in the direction of a main axis (21) of the implant (20, 70) and which are interconnected to one another by struts (12); wherein the struts (12) between two adjacent circumferential line segments (11) are inclined in the same direction and wherein the inclination direction of struts (12) arranged below a circumferential line segment (11) is opposite to the inclination direction of struts (11) arranged below that circumferential line segment (12). A corresponding method for the manufacture of the implant (20, 70) according to various embodiments is also provided.

In various embodiments the present invention relates to a biodegradable implant. The implant according to various embodiments may be a bone implant and be filled with a substance, such as biological cell material, such as graft material from a subject, and/or synthetic material and be transplanted into the subject as replacement for a portion of a bone. The present invention further relates to a method for the manufacture of the implant according to various embodiments.

The insertion of implants into patients is performed in surgeries worldwide. Depending on the required use of the implant, an implant may be required to meet certain criteria. Such criteria may relate to the geometry of the implant and/or the mechanical stability of the implant. In some instances, such as wherein an implant may be used in reconstructive surgery, the implant may have to be handled by a surgeon before, during and/or after the implant is inserted into a patient. It is desirable to have an implant which meets the geometrical and mechanical criteria required by the patient, and which is easily handled and/or manipulated by surgeons performing implant surgery.

In various embodiments, an implant is provided which may be constructed to meet criteria required in a reconstruction performed on a subject's body, in particular in replacing missing bones or bone portions. The implant according to various embodiments may be provided in various shapes, which all follow the inventive concept, to provide a supporting scaffold which contains the substance to be transplanted in a desired three-dimensional form. In that sense, the inventive implant may be also referred to as a “bone (material) cage”. At the same time, once implanted, the implant according to various embodiments aims at providing an environment conducive to the transformation of the transplanted substance into solid bone. Finally, the implant according to various embodiments may also provide improved on-site handleability and manipulability thereof by surgeons performing the surgery. In particular, the implant according to various embodiments has a configuration which allows easy adjustment of its axial dimension by the surgeon during the surgery without affecting the advantageous dynamic properties of the implant.

In various embodiments, an implant for containing a substance, such as graft material, which may comprise biological cell material or synthetic bone graft material, is provided. Synthetic bone graft material may comprise bone cement and/or calcium phosphate/sulphate and/or hydroxyapatite. The substance may also comprise growth factors as an addition to the other substances or just growth factors which are comprised in a bulk material, sch as a holding matrix. The biological cell material may comprise bone marrow optionally including further substances, such as growth factors, which may be added to the biological cell material, which is filled into the implant, to enhance the transformation of the graft material into solid bone. Before being implanted, the substance be introduced into the implant through the mesh which may be seen to correspond to a porous outer wall of the implant. Therefore, due to this function, the implant according to various embodiments may be also referred to as a graft material cage.

The implant according to various embodiments may be manufactured from a biodegradable material which, over time, may be decomposed by the body of the subject. Preferably, the implant according to various embodiments may be manufactured by means of 3D printing.

The implant according to various embodiments comprises a mesh (scaffold) formed into a continuous sidewall. The sidewall may correspond to a sleeve which extends continuously around a main axis of the implant. Expressed differently, the mesh, which corresponds to the sidewall of the implant, forms a closed poly gonal outer surface without axial gaps in the sidewall. The main axis denotes the axial direction of the implant and may but does not necessarily have to be located at the center of the top or bottom area of the implant. In the case of a circular bottom area of the implant, the main axis may extend from or run through the center of the bottom area. In the case of a substantially round bottom area with an irregular circumference, the main axis may extend from or run through a point which is close to the center of gravity of the bottom area. In both cases, the main axis may run through or extend from a point which lies within the perimeter of the bottom surface and/or the perimeter of the top surface of the implant. The main axis may be an axis along which the height of the implant may be measured. Overall, some embodiments of the implant may have a tubular or cylindrical shape, wherein the top and the bottom of the implant do not necessarily have to have the same or similar shape. This means that the implant according to various embodiments may have a slanted side wall and, to a first approximation, look like a truncated cone, where the top and bottom surfaces may but do not necessarily have to be aligned centrally relative to one another. In further embodiments, the uppermost and lowermost circumferential line segments may have a sickle-or C-shape with rounded ends.

Implants having that shape may be used when a gap in a bone into which the implant is to be inserted is supported with a bone nail. In that case, the concave shape of the implant may be placed tightly around the bone nail.

The mesh of the implant according to various embodiments comprises a plurality of circumferential line segments, which are arranged above one another in the direction of the main axis (axially) and which are interconnected to one another by struts. The circumferential line segments are formed as closed shapes and their shapes may gradually change from top to bottom (or vice versa). The circumferential line segment arranged at the bottom of the implant corresponds to the perimeter of the bottom surface of the implant. In the same manner, the circumferential line segment arranged at the top of the implant corresponds to the perimeter of the top surface of the implant. Starting with the circumferential line segment at the bottom of the implant, each of the circumferential line segments is connected to the circumferential line segments arranged above by means of a plurality of struts. However, each of the circumferential line segments may have a form which is different from its adjacent (one below and one above) circumferential line segments. In particular, the circumferential line segments may be formed such that a gradual transition from the bottom area to the top area of the implant can be achieved. The struts used to establish a mechanical interconnection between the circumferential line segments may correspond to straight line segments (lines) or bars.

The implant according to various embodiments is configured such that the struts between two adjacent circumferential line segments are inclined in the same direction and wherein the inclination direction of struts arranged below a circumferential line segment is opposite to the inclination direction of struts arranged below that circumferential line segment. The angles between the struts extending upwards from a respective circumferential line segment and that circumferential line segment are, by definition, all larger than 0° and smaller than 90° (obviously, the same applies to the angles between the struts extending downwards from a respective circumferential line segment and that circumferential line segment), wherein the struts are either slanted to the left or to the right when the implanted is viewed in a side view, i.e. perpendicularly to its main axis.

The slanted arrangement of the struts between two circumferential line segments has the effect that, when pressure (load) is applied to the implant from the top or from the bottom, the struts act as elastic elements and to some extent bend such that the angels between the struts and the circumferential line segment may grow smaller. That is, by trying to squeeze an exemplary implant comprising only two circumferential line segments which are interconnected by struts, which, without loss of generality, are slated to the left when the implant is viewed from outside, the struts will be further bent to the left which will lead to the two circumferential line segments moving closer together. At the same time, both circumferential line segments will rotate with respect to one another. Therefore, when a load is applied on the implant from top or from the bottom the implant according to various embodiments will behave as if it were loaded in torsion and a respective circumferential line segment will rotate with respect to the adjacent circumferential line segments while keeping its general orientation relative to the other circumferential line segments. Expressed differently, the struts arranged between two consecutive circumferential line segments act, mechanically, as spring elements allowing the two consecutive circumferential line segments to move closer to one another, which at the same time rotate against one another, when an axial load is applied to the implant. When the load is removed, the struts return to their initial orientations/configurations of being straight segments and the circumferential line segments move apart from one another, again rotating against one another, but in the opposite direction. In that manner, the force acting on the implant may be seen as being stored in torsion of the implant which alternates from one circumferential layer of the implant to the next. In the context of this description, a circumferential layer of the implant is understood as two consecutive circumferential line segments and the struts connecting those two. Each circumferential layer may be viewed as a level of the implant. The implant according to various embodiments comprises at least one circumferential layer, wherein the distance between two consecutive circumferential line segments in an unloaded state of the implant comprising at least two circumferential layers can be the same or different.

In an implant according to various embodiments comprising a plurality of circumferential line segments the alternating inclination direction of the struts from one level to the next level of the implant will lead to an alternating change in the rotation direction of a respective circumferential line segment relative to its upper and lower circumferential line segment. This has the effect that, when a pressure is applied on the implant from top or from the bottom (i.e. the implant is being squeezed together by a force acting along its main axis), the distance between the circumferential line segments will decrease, while the pressure will be “stored” in the torsion of the circumferential layers. In other words, the rotation direction of the circumferential line segments will alternate from one circumferential line segment to the next. The implant according to various embodiments may be seen to correspond to a structure which has a zero Poisson's ratio, i.e. a structure which does not exhibit lateral deformation when it is squeezed by an axial load(from the top or from the bottom or both).

In more detail, in experiments the implant according to various embodiments has been found to show very little or negligible lateral expansion when compressed, at the same time preserving its initial structure after may load cycles. Expressed differently, in a cross-sectional side view, i.e. viewed perpendicularly to the axis of load, the implant of the present invention substantially does not show buckling, which is understood as a disadvantageous lateral deformation thereof when compressed by a load acting along the main axis of the implant. This may be seen as a medically advantageous behavior of the implant according to various embodiments because the implant is able to provide an environment to the bone graft material which is similar to a natural environment in which axial loads acting on a broken bone assist the healing process and lead to the formation of solid bone at the injured site.

According to various embodiments of the implant, the circumferential line segments and the struts may comprise an elastic material. As noted above, the elastic material may, in particular, be one which is 3D printable. The elastic material used for the circumferential line segments and the struts may be the same or different. For example, the circumferential line segments may comprise a material which has a higher stiffness than the material of the struts. In that manner, the configuration of the implant may correspond to a structure in which relatively sturdy circumferential line segments are connected to one another by relatively soft struts which act as elastic elements allowing the circumferential line segments to move, without deformation, closer to one another when a load acting along the main axis of the implant is applied thereto. Such a dual-material implant according to various embodiments may be manufactured using a dual extruder 3D printer.

According to various embodiments of the implant the struts between two adjacent (consecutive) circumferential line segments may be inclined at the same inclination angle. At the same time, the struts between two adjacent circumferential line segments may have the same length and, preferably, the same dimension. Such a configuration of the struts may lead to an implant which may be said to comprise unit cells in the form of parallelograms which are arranged one adjacent to the other between two circumferential line segments. In an implant according to various embodiments comprising a plurality of circumferential layers, the configuration of the struts of a respective circumferential layer does not have to be the same as the configuration of the struts of the other circumferential layers. It is thus possible to have circumferential layers with unit cells of different sizes in order to adjust the dynamic behavior of the implant.

Generally, in dependent of the actual value of the inclination angle and the actual configuration of the unit cells (all the same or different ones in each circumferential layer), the implant according to various embodiments comprises a configuration which allows reversible collapse or closing of the unit cells from a quiescent state of the implant to a compressed state of the implant (and back to the quiescent state once the load is removed).

According to various embodiments of the implant the inclination angle of struts arranged above a respective circumferential line segment may be the same as the inclination angle of struts arranged below that circumferential line segment. Alternatively, the inclination angle of struts arranged above a respective circumferential line segment may be different from the inclination angle of struts arranged below that circumferential line segment. In general, the inclination angle of the struts arranged between two consecutive circumferential line segments is a design parameter which may be adjusted, in combination with other design parameters of the implant, in order to obtain an implant with a predefined stiffness/rigidity.

According to various embodiments of the implant, the inclination angle of the struts may lie between approximately 10° and approximately 80°, preferably between approximately 20° and approximately 70°, more preferably between approximately 30° and approximately 60°, more preferably between approximately 40° and approximately 50°. As a general rule of thumb, it has been found that smaller inclination angles yield softer implant structures.

According to various embodiments of the implant, the number and/or the size of the of struts arranged above a respective circumferential line segment may be the same as the number and/or the size of the of struts arranged below that circumferential line segment. The size of a strut may refer to its length and/or its diameter (or circumference, in case the struts do not have a tubular shape). Alternatively, the number and/or a size of the of struts arranged above a respective circumferential line segment may be different from the number and/or the size of the of struts arranged below that circumferential line segment. In that manner, the dynamic behavior of the implant under load can be fine-tuned, level by level.

According to various embodiments of the implant, the struts arranged between two respective circumferential line segments may be provided at equal distances from one another. In other words, the struts between to circumferential line segments may be arranged equidistantly along the perimeter of the implant.

According to various embodiments of the implant, at least one anchor point of a strut connecting to a respective circumferential line segment from below may be aligned with or may coincide with an anchor point of a strut connecting to that circumferential line segment from above. Preferably, all anchor points of struts connecting to a respective circumferential line segment from below may be aligned or may coincide with anchor points of struts connecting to that circumferential line segment from above. Such a configuration of an implant is characterized by continuous zig-zag lines, formed by the struts of consecutive circumferential layers, which run between the uppermost and lowermost circumferential line segment and interconnect the circumferential line segments lying in between.

In an alternative to the previous embodiments, according to various further embodiments of the implant the anchor points of struts connecting to a respective circumferential line segment from below may be shifted relative to the anchor points of struts connecting to that circumferential line segment from above. Expressed differently, the anchor point of a strut connecting to a respective circumferential line segment from above may lie between two anchor points of struts connecting to that circumferential line segment from below (or vice versa). The location of the anchor points of the struts is a design parameter which may be adjusted to adjust the point between two anchor points of two struts connecting to that circumferential line segment from below, for example, at which a bending moment, induced by a strut connecting to a respective circumferential line segment from above, acts on the respective portion of the circumferential line segment. Here, the portion of the circumferential line segment between the anchor points of two struts connecting to that circumferential line segment from below may be seen to correspond to a beam which is supported at both ends by the two struts and which is subjected to a bending moment induced by the strut connecting to the circumferential line segment from above. This design parameter alters the dynamic behavior of implant when a load acting along the main axis of the implant is applied thereto.

According to various embodiments the implant may have a Poisson's ratio of approximately zero. This is achieved by the configuration of the implant which features levels, arrangements of struts between two circumferential line segments, thus allowing the implant to absorb axial load and store it in elastic bending of the struts and an overall rotation of the levels with respect to one another. Instead of lateral bloating under axial strain, the circumferential line segments of the implant according to various embodiments rotate with respect to one another and decrease their distance due to bending of the elastic struts. Once the axial load is removed, the implant returns to its quiescent state, while substantially preserving its cross-sectional shape.

According to various embodiments of the implant a bottom circumferential line segment and/or a top circumferential line segment may each define a contour which matches a contour of a corresponding portion of the bone which is to be contacted by the bottom and the top of the implant. Such a configuration allows for an optimal interface between the implant and the portions of the bone which surround the implant in the sense that there is substantially no discontinuity between the outer lateral surface of the bone and the outer lateral surface of the implant. Therefore, the implant according to various embodiments may be manufactured, in particular 3D printed, as a custom implant which fits exactly into a gap in a bone of a subject by adjusting the form of its top and bottom.

According to various embodiments of the implant each of the circumferential line segments may define a plane which is arranged perpendicularly to the main axis of the implant.

In various embodiments, the present invention further relates to an implant for use in bone reconstruction or bone augmentation surgery, in which the implant according to various embodiments filled with the substance, as defined above, is implanted into a subject.

Furthermore, in various embodiments the present invention provides a method for manufacturing an implant for containing the substance, as defined above, the method comprising forming a plurality of circumferential line segments interconnected by struts to obtain a mesh formed into a sidewall of the implant, wherein the mesh comprises a plurality of circumferential line segments, which are arranged above one another in the direction of a main axis of the implant and which are interconnected to one another by struts; wherein the struts between two adjacent circumferential line segments are inclined in the same direction and wherein the inclination direction of struts arranged below a circumferential line segment is opposite to the inclination direction of struts arranged below that circumferential line segment.

Furthermore, in various embodiments the present invention provides a method for adjusting the size of an implant containing a substance, the method comprising removing at least one circumferential line segment and the struts attached thereto to obtain an implant with a reduced axial dimension. Exploiting the highly symmetric and repetitive configuration of the circumferential layers formed by two consecutive circumferential line segments with struts arranged therebetween, the height of the implant may be adjusted on site by the surgeon by cutting off at least one of the lowermost and/or uppermost circumferential line segments together with the struts attached to it. The final height adjusted implant is again an implant in which the lowermost and uppermost element is a circumferential line segment. Advantageously, the general dynamic behavior of the implant is not affected by the size adjustment. It is noted that in the context of this description, the height of the implant is understood as its dimension along its main axis.

Finally, the present invention also relates to a method of bone reconstruction or bone augmentation surgery, wherein the method comprises filling an implant according to various embodiments disclosed herein with the substance, as defined above, and implanting the filled implant into the body of a subject.

The foregoing and other features of the present disclosure will become more fully apparent from the following description and appended claims, taken in conjunction with the accompanying drawings. The accompanying drawings illustrate only several embodiments in accordance with the present invention and are, therefore, not to be considered as limiting its scope. Embodiments of the invention will be described with additional specificity and detail with reference to the drawings, such that the advantages of the present invention can be more readily ascertained, in which:

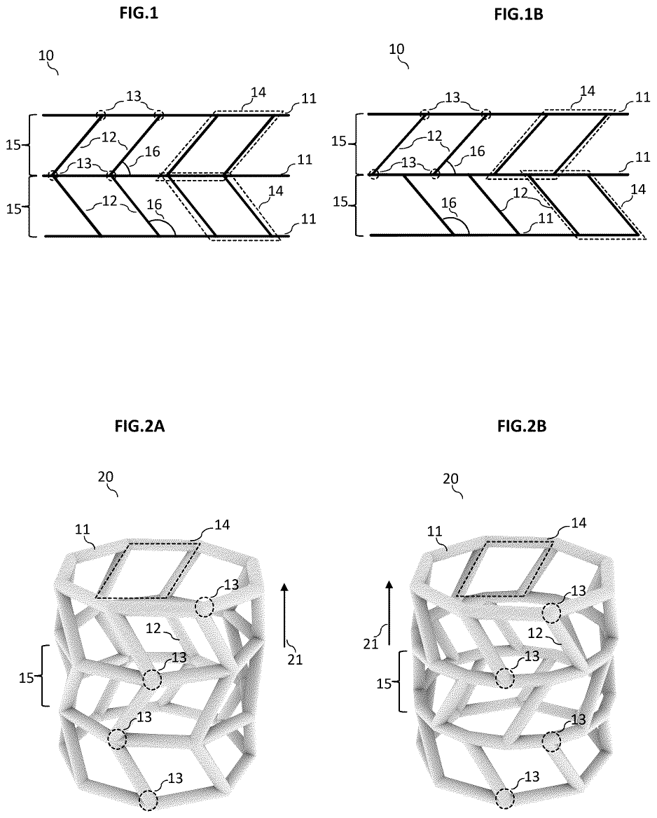

FIGS. 1A and 1B shows embodiments of the circumferential layers of the implant according to various embodiments.

FIGS. 2A and 2B shows embodiments of the implant with configurations of the circumferential layers as shown in FIGS. 1A and 1B, respectively.

FIGS. 3A and 3B illustrate the behavior of implants according to various embodiments under compression.

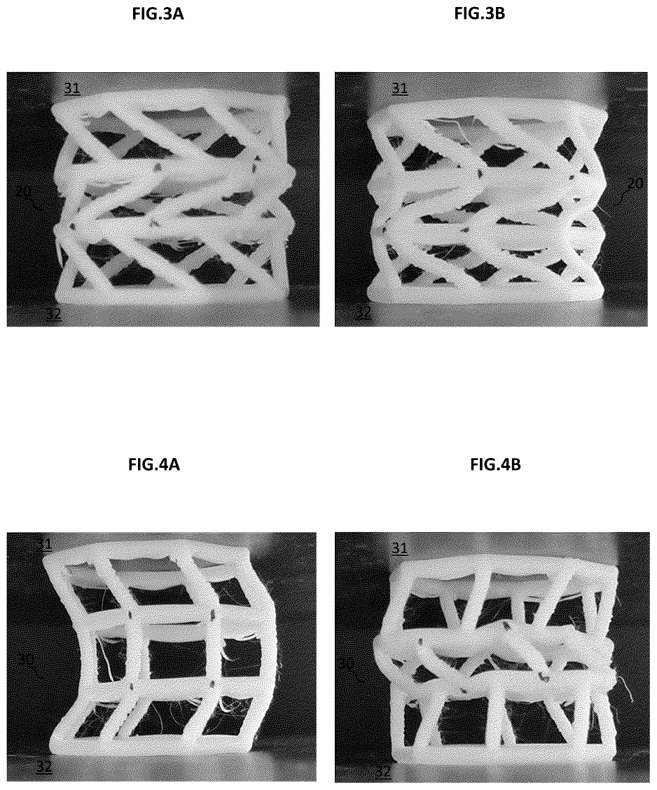

FIGS. 4A and 4B illustrate the behavior of an ordinary implants with a rectangular mesh as outer wall under compression.

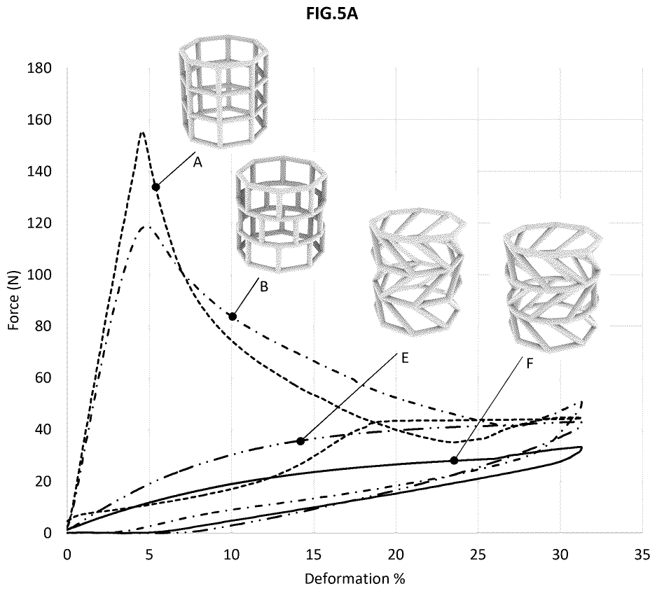

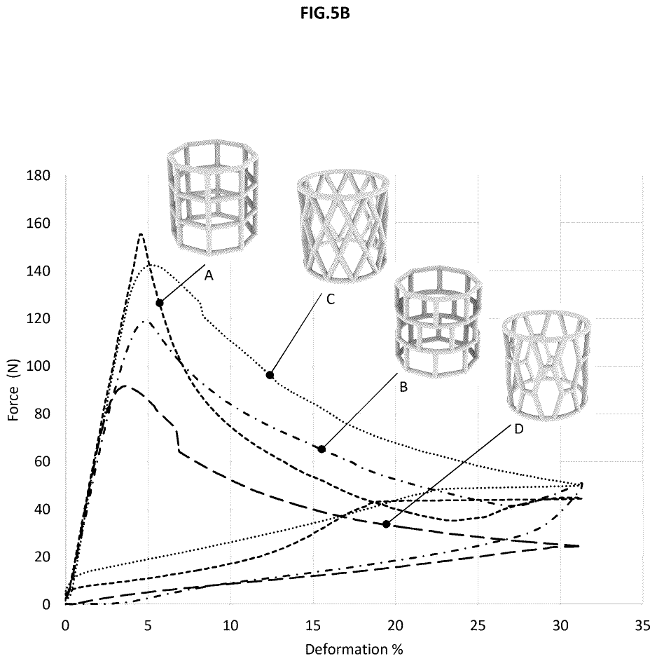

FIGS. 5A-5C shows diagrams in which deformation-force curves for various configurations of the implant according to various embodiments under axial load are shown, compared to ordinary implants.

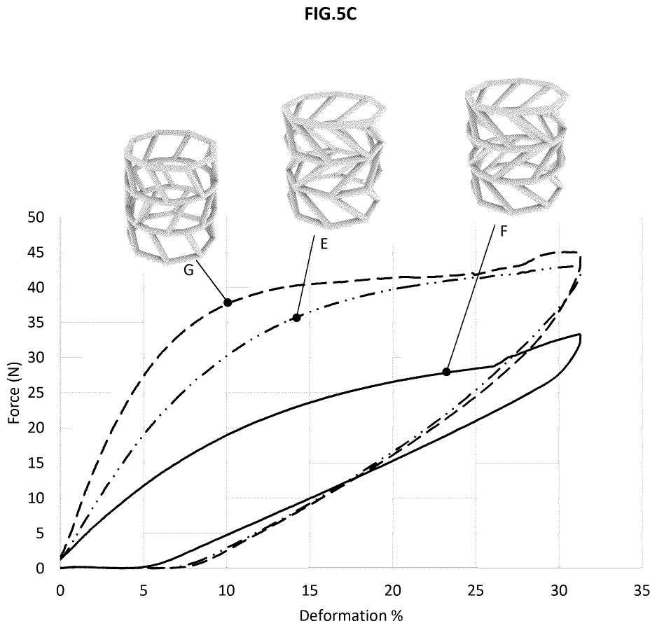

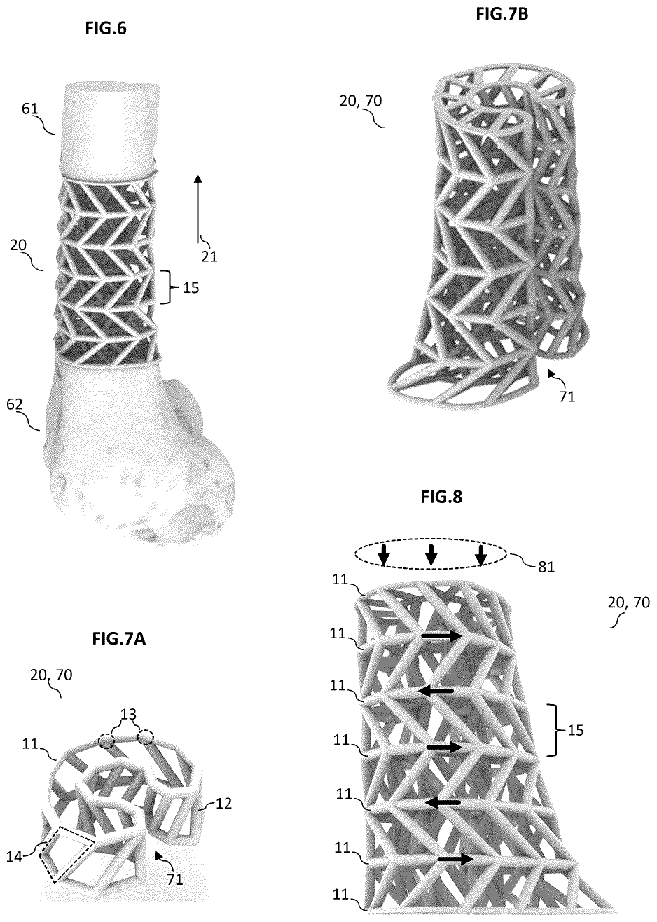

FIG. 6 illustrates the use of the implant according to various embodiments for bone reconstruction.

FIG. 7A shows a C-shaped implant according to various embodiments.

FIG. 7B shows an implant according to various embodiments, which is based on the C-shaped form shown in FIG. 7A.

FIG. 8 illustrates the distribution of forces within the implant according to various embodiments under axial load.

In the following detailed description, reference is made to the accompanying drawings that show, by way of illustration, specific embodiments in which the claimed subject matter may be practiced. These embodiments are described in sufficient detail to enable those skilled in the art to practice the subject matter. It is to be understood that the various embodiments, although different, are not necessarily mutually exclusive. The terms “embodiment,” “example embodiment,” “exemplary embodiment,” and “present embodiment” do not necessarily refer to a single embodiment, although they may, and various example embodiments may be readily combined and/or interchanged without departing from the scope or spirit of example embodiments. For example, a particular feature, structure, or characteristic described herein, in connection with one embodiment, may be implemented within other embodiments without departing from the spirit and scope of the claimed subject matter. References within this specification to “one embodiment” or “an embodiment” mean that a particular feature, structure, or characteristic described in connection with the embodiment is included in at least one implementation encompassed within the present description. Therefore, the use of the phrase “one embodiment” or “in an embodiment” does not necessarily refer to the same embodiment. In addition, it is to be understood that the location or arrangement of individual elements within each disclosed embodiment may be modified without departing from the spirit and scope of the claimed subject matter. The following detailed description is, therefore, not to be taken in a limiting sense, and the scope of the subject matter is defined only by the appended claims, appropriately interpreted, along with the full range of equivalents to which the appended claims are entitled. In the drawings, like numerals refer to the same or similar elements or functionality throughout the views, and elements depicted therein are not necessarily to scale with one another, rather individual elements may be enlarged or reduced in order to more easily comprehend the elements in the context of the present description.

The terms “a,” “an,” and “the” may include singular and plural references. Furthermore, as used in the present disclosure and the appended claims, the words “and/or” may refer to and encompass any and all possible combinations of one or more of the associated listed items. As used in the present disclosure, the phrase “A and/or B” means (A), (B), or (A and B). As used in the present disclosure, the phrase “A, B, and/or C” means (A), (B), (C), (A and B), (A and C), (B and C), or (A, B and C). As used in the present disclosure, term “or” when used in the phrase “A, B, or C” means that (A) does not exclude (B) and (C), (B) does not exclude (A) and (C), and (C) does not exclude (A) and (B).

FIGS. 1A and 1B show embodiments of circumferential layers 15 comprised by the mesh 10 of the implant according to various embodiments which makes up its sidewall. In both figures, the same elements are labelled with the same reference numbers. In both figures, the mesh 10 comprises two circumferential layers 15. Each circumferential layer 15 comprises two circumferential line segments 11, which are arranged above one another in the direction of the main axis, and which are interconnected to one another by struts 12. The struts 12 correspond to bars or rods which establish interconnections between the circumferential line segments 11. The struts 12 connect to the circumferential line segments 11 at anchor points or connection points 13. Each of the circumferential layers 15 comprises a plurality of unit cells 14 which are arranged one adjacent to the other. The unit cells 14 are also arranged one adjacent to the other vertically, when different circumferential layers are considered. In both embodiments of the portions of the mesh 10 the struts 12 between two adjacent circumferential line segments 11 are all inclined in the same direction and the inclination angle 16 of the struts 12 within one circumferential layer 15 is the same. However, the inclination direction of struts 12 arranged below the middle one of the three shown circumferential line segments 11 is opposite to the inclination direction of struts 12 arranged below that circumferential line segment 11. That is, in both embodiments, the struts 12 of the first circumferential layer 15, counted from bottom to top, are slanted to the left side, while the struts 12 of the second circumferential layer 15, which is arranged on the first circumferential layer 15, are slanted to the right side. The inclination angle 16 between the struts 12 and the line segment 11 is a design parameter which may be changed to alter the mechanical property of the implant 20.

The two embodiments of the mesh 10 shown in FIGS. 1A and 1B differ from one another in that in FIG. 1A the anchor points 13 between the struts 12 and the circumferential line segments 11 of the two circumferential layers 15 coincide. That is, the anchor points 13 of struts arranged above a respective circumferential line segment 11 (will be referred to as upper struts in the following), in this case the middle one, and connecting to it correspond to the anchor points 13 of struts 12 arranged below that circumferential line segment 11 (will be referred to as lower struts in the following) and connecting thereto. On the contrary, in the embodiment of the mesh 10 show in FIG. 1B, the anchor points 13 of struts 12 arranged above the middle circumferential line segment 11 and connecting to it do not coincide with the anchor points 13 of struts 12 arranged below that circumferential line segment 11 and connecting thereto. In fact, as shown in FIG. 1B, the anchor points 13 of the upper struts 12 are arranged midway between the anchor points 13 of the lower struts 12. In other words, in the embodiment shown in FIG. 1B, the circumferential layers 15 seem to be rotated with respect to one another by an arc which corresponds to the shift between the anchor points of the 13 upper and lower struts 12 compared to the embodiment shown in FIG. 1A.

Embodiments of the implant which are based on the meshes 10 from FIGS. 1A and 1B are illustrated in FIGS. 2A and 2B, respectively. Each of the implants 20 comprises three circumferential layers 15 which are stacked on top of one another in the direction of the main axis 21 of the implants 20. Each of the implants 20 has, to first approximation, a cylindrical or tubular shape. This means that the mesh 10, which forms the sidewall of the implants 20, is in itself continuous in that it forms a laterally closed surface around the interior of the implant 20 and that the circumferential line segments 15 are closed geometrical forms-in the exemplary case shown in FIGS. 2A and 2B rounded polygons. The pores in the form of unit cells 14 in the mesh 10 are neglected in the assessment whether the sidewall of the implant 20 is closed or not. The further elements which may be seen in the 3D representations of the implants 20 have already been described with reference to FIGS. 1A and 1B and hence will not be repeated.

FIGS. 3A and 3B show actual images of the implant 20 according to various embodiments which illustrate the behavior of the implant 20 under compression. Both embodiments of the implant 20 chosen for the experiment have a diameter of 3 cm and has a height of 3 cm, such that each of the three circumferential layers 15 has a height of approximately 1 cm in the quiescent state of the implant 20. In the embodiment of the implant 20 in FIG. 3A the anchor points of the struts above a circumferential line segment are shifted to the midpoints between the anchor points of the struts below a circumferential line segment. In the embodiment of the implant 20 in FIG. 3B the anchor points of the struts above a circumferential line segment are aligned with the anchor points of the struts below a circumferential line segment. Both implants 20 are placed between an upper plate 31 and a lower plate 30 and loaded with a compression force acting along the main axis of the implants 20. In both figures, the implants 20 according to various embodiments has been compressed by approximately one third of their axial dimension.

As can be taken from the images shown in FIGS. 3A and 3B, the scaffold of both embodiments of the implant 20 is able to absorb the load without buckling, i.e. while preserving its cross-sectional shape. The load is absorbed by bending of the struts 12 in their biased direction, i.e. such that the inclination angle is decreased. While the circumferential layers 15 rotate in the opposite direction with respect to one another, the uppermost and the lowermost circumferential line segments 11 (top and bottom of the implant 20) remain fixed due to their friction against the upper plate 31 and lower plate 32. Furthermore, it can be seen that the cross-sectional shape of both embodiments of the implant 20 remains practically the same, as the struts do not bend outwardly beyond the perimeter delimited by the circumferential line segments.

For comparison, the same experiment has been conducted with two implants 30 which do not feature the inventive configuration and thus will be referred to as “ordinary implants 30” in the following. Merely for ese of reference and understanding, structurally same elements will be denoted using the terminology which has been used to describe the implants 20 of the present invention. The images depicting the progressive deformation of the ordinary implants 30 are shown in FIGS. 4A and 4B, wherein for better comparison to the implant according to the invention shown in FIGS. 3A and 3B the ordinary implants 30 are also 3 cm high and also have a bottom and top surface with a diameter of 3 cm. The scaffold of the ordinary implant 30 has circumferential line segments, but its struts and consequently also the unit cells are different in that the scaffold features a quadratic pattern, where the struts are not laterally biased or, so to speak, but stand erect. In other words, using the terminology which has been used to describe the inventive implant 20, the ordinary implant 30 comprises struts which extend from or connect to the circumferential line segments at right angles. In analogy to FIGS. 3A and 3B, the ordinary implant shown in FIG. 4A the anchor points of the struts above a circumferential line segment are shifted to the midpoints between the anchor points of the struts below a circumferential line segment. In the ordinary implant 30 shown in FIG. 4B the anchor points of the struts above a circumferential line segment are aligned with the anchor points of the struts below a circumferential line segment. The loads acting upon the ordinary implants 30 in FIGS. 4A and 4B are the same as the loads acting upon the implants 20 according to various embodiments shown in FIGS. 3A and 3B.

As can be seen in FIG. 4A, the ordinary implant 30 with aligned anchor points of the struts suffers severe buckling-it bends sideways in its entirety, away from the axis of the applied load, and is therefore not able to preserve its cross-sectional shape. In FIG. 4B the situation is even worse, as the ordinary implant 30 with shifted anchor points is permanently deformed. While a portion of the upper circumferential line segment of the first circumferential layer (counting from the bottom upwards) bends inwards, a corresponding portion of the lower circumferential line segment of the third circumferential layer bends outwards, such that the middle level of the ordinary implant 30 is heavily deformed. Contrary to the inventive implant 20 shown in FIGS. 3A and 3B, the ordinary implant 30 does not have the ability to absorb the load by a coordinated and substantially homogenous elastic deformation of its scaffold.

By comparing the behavior of the implant 20 according to various embodiments to the behaviour of the ordinary implant 30 which has not been constructed in accordance with the invention, it may be seen that, surprisingly, the arrangement of the alternatingly slanted struts 12 (or, consequently, the slanted unit cells 14) in subsequent circumferential layers 15 provides an inventive scaffold of the implant which is able to not only keep its cross-sectional shape (no buckling), but can also accept a relatively large load leading to a reversible deformation without failing. In the case of the ordinary implant 30 in FIG. 4B, on the contrary, once the load has been removed, the implant 30 was not able to return back to its initial state and has thus suffered permanent damage rendering it dysfunctional for the intended purpose of bone reconstruction.

The exemplary results illustrated in FIGS. 3A, 3B and 4A, 4B have been examined more systematically by applying deformation to various configurations of the inventive implant and ordinary implant and observing their behavior. In the diagrams shown in FIGS. 5A-5C the amount of deformation force (y-axis), in Newton, applied to an implant is graphed over the resulting deformation (x-axis), in percent of the height of the implant, for various configurations of the implant. A deformation of 10% means that the distance between plates pressing on the respective implant (see FIGS. 3A,3B and 4A, 4B) and deforming it has been reduced by an amount which corresponds to 10% of the height of the implant in its quiescent state. The force on the y-axis describes the resulting load acting on the implant at a certain level of deformation. All the implants which have been examined in the conducted deformation experiment had a tubular or cylindrical shape with a top and bottom diameter of 3 cm and a height of 3 cm. In the conducted experiments, a deformation of 33% has been applied to all the implants and then the compressing plates have been driven apart again while measuring the force exerted by the compressed implant. Hence all the curves in the diagrams 5A-5C have an end point at a deformation slightly above 30%. It is noted that the deformation applied in the conduced experiments was achieved by compressing the implants along their main axis and hence the deformation experiments corresponded to compression experiments.

In the diagram in FIG. 5A, curves A and B belong to ordinary implants which do not feature the inventive configuration. Curve A describes the deformation-force relation for the ordinary implant 30 shown in FIG. 4A and curve B describes the deformation-force relation for the ordinary implant 30 shown in FIG. 4B.

As can be seen by observing curves A and B, the corresponding implants representing an ordinary design reach their maximum capacity with respect to load acceptance at relatively low deformations just below 5%, characterized by pronounced peak and decreasing force values for larger deformations. Increasing the deformation past the peaks led to irreversible deformations of the ordinary implants characterized by cracked struts. Compressing an ordinary implant further after it has reached its peak on the deformation-force curve led to mechanical failure of the structure which manifested itself in loss of mechanical stability in the form of cracks in the structure or even breaking and delamination, i.e. layer separation in the 3D printed implant scaffold.

Already from a mere qualitative comparison of the other two curves E and F it may be immediately seen that those deformation-force curves belong to different kind of implants featuring superior behaviour when deformation is applied to them. The deformation-force curves E and F have been obtained based on implants according to various embodiments of the invention and they clearly indicate that the corresponding implant structures have an improved deformation acceptance capability in that even at a 30% deformation they have not reached their peak force (which would indicate the maximum capacity, just as in the case of the curves A and B) and thus can withstand more deformation without permanent damage. This may be taken from the fact that while the ordinary implants (curves A and B) reach their global force maximum at a deformation slightly below 5%, the implants of the present invention (curves E and F) do not. The maxima reached by curves E and F would be seen to correspond local maxima if the experiment would be conducted towards larger deformations that slightly over 30%. The implants according to the invention deform gradually under the influence of compression force and, consequently, the curves A and B rise continuously up to the end point of the experiment. In contrast to the ordinary implants, which reach their global maximum quite early on and thus do not have a continuously rising deformation-force curves, the implants according to the invention remain in elastic regime over the entire deformation region of the conducted compression experiment. Most particularly, in direct comparison to the ordinary implants, the embodiments of the implant according to various embodiments of the invention did not experience any irreversible deformations in the conducted experiments over many cycles (close to a thousand) of applied deformation.

Overall, the results presented in the diagram of FIG. 5A imply that the implants according to various embodiments are softer than the ordinary implants as the same amount of applied compression force leads to a larger deformation as compared to the ordinary implants. In detail, curve E describes the deformation-force relation for an implant according to various embodiments with coinciding anchor points 13 for upper and lower struts 12 and an inclination angle 16 of 40°. Curve F describes the deformation-force relation for an implant according to various embodiments, with non-coinciding locations of the anchor points 13 for upper and lower struts 12 and an inclination angle 16 of 40°. Thus, by comparing curve E to curve F, the influence of coinciding and non-coinciding anchor points on the dynamic behaviour of the implant according to various embodiments can be analyzed.

Furthermore, based on the differences observed in the diagram of FIG. 5A between the deformation-force relations of ordinary implants (curves A and B) and the implants according to various embodiments of the invention (curves E and F) and in view of the visually different reaction of the scaffolds to compression as visualized in FIGS. 3A, 3B and 4A, 4B, the implant of the present invention has the advantage that a load applied to it leads to a more uniform distribution of the applied load on the circumferential layers. As can be taken from a comparison of FIG. 3B to FIG. 4B, in the implant 20 according to the invention every circumferential layer is compressed to some extent, i.e. the upper and lower circumferential line segments 11 of each circumferential layer are moved closer to one another. On the contrary, in the ordinary implant 30 shown in FIG. 4B, the first and third circumferential layers, counting from bottom to top, experience little compression, while the entire load seems to act on the middle circumferential layer which is deformed into failure. Thus, as can be taken from FIGS. 4A and 4B, the ordinary implant 30 displays a very inhomogeneous compression pattern, which manifests itself in the sharp spike in the deformation-force curves A and B. Considering the intended use of the implant according to various embodiments, it may be seen that the inventive implant 20 offers an advantageous, relatively homogeneous compression profile. Namely, after implantation into the subject, the substance contained in the implant 20 experiences compressive forces throughout the entire implant 20, which is important for providing nutrition to the substance, vascularization and transformation of the initially relatively soft substance into a solid bone structure. The deformation-force curves A, B and E, F obtained in FIG. 5A are based on a single cycle including a deformation phase where deformation is applied to the implants and a release phase where the applied deformation is released and the implants decompress. Even though all four deformation-force curves imply that all four implants return back to their initial state in the release phase, it is to be pointed out that the ordinary implants have suffered irreversible damage and seem to return to their initial state driven by the elastic force of the material of the scaffold, but without structural integrity. This means that in a subsequent cycle the ordinary implants A and B are structurally compromised and will feature fundamentally different deformation-force curves. This renders the ordinary implants A and B unusable for cyclic deformation. Under cyclic deformation, the defects suffered by the ordinary implant in a previous cycle will aggravate, leading to complete failure of the ordinary implant after a few cycles.

The embodiments A and B of the implant according to the invention maintain their structural integrity and feature the same deformation-force curves over several hundreds of deformation cycles. This quality may be attributed to the fact that for the inventive implants the force peaks in the deformation-force curves lie beyond the 30% deformation mark and therefore the inventive implants operate in their elastic regime where the deformation may be stored in the scaffold and released therefrom for very many cycles without the implant scaffold suffering any damage. This may be particularly advantageous for the intended use of the inventive implants in bone replacement therapy where after insertion, the implant will be compressed into a preloaded state which becomes its biomechanical quiescent state and will experience dynamization, i.e. further compression and decompression around that preloaded quiescent state. For example, an implant with an axial dimension of 3 cm may be assumed to be deformed to a preloaded axial dimension of 2.5 cm and to oscillate between 0.3 cm and 0.7 cm (e.g. corresponding to cyclic deformation of a walking patient with the inventive implant located in his femur) from that preloaded state. As evidenced by the deformation-force curves E and F, the inventive implant may provide the required dynamization in the range of 0.3-0.7 cm over a long period of time without suffering structural damage.

In FIG. 5B, the deformation-force curves of two further ordinary implants are compared with deformation-force curves of the ordinary implants from the diagram of FIG. 5A. Namely, curve C describes the deformation-force relation for an ordinary implant which comprises a mesh having diamond-shaped unit cells, while curve D describes the deformation-force relation for an ordinary implant which comprises a mesh having hexagonal unit cells. As can be seen from the comparison of the curves, qualitatively the ordinary implants with diamond-shaped unit cells and with hexagonally shaped unit cells show the same behaviour as the other two ordinary implants which have been already described with reference to the diagram of FIG. 5A. Both curves C and D reach their global maximum at or slightly below 5% deformation and all the curves feature a highly asymmetric nature in the sense that the “upper” portion of each curve is fundamentally different from the corresponding “lower” portion thereof.

The diagram in FIG. 5C shows the curves E and F from the diagram of FIG. 5A together with a further deformation-force curve G for an implant according to the invention as shown in FIG. 2B, i.e. with separate or non-coinciding anchor points for upper and lower struts of a respective circumferential line segment and an inclination angle of 70°. As can be seen, the range on the y-axis of the diagram in FIG. 5C has been decreased to 0-50 N as compared to the y-axis in the diagram of FIG. 5A which covers a range of 0 -180 N in order to show the deformation-force curves E, F, and G of embodiments of the inventive implant in more detail. All the embodiments of the inventive implant used in the compression experiment have the same height of 3 cm, diameter of 3 cm and number of struts. Also, the amount of material used for the manufacture of the embodiments of the inventive implant was roughly the same. From a comparison of curve F to curve G, it may be clearly seen that the softness of the implant according to the invention may be decreased by increasing the inclination angle of the struts. In addition, the obtained results imply that a softer implant (curve F) seems to have a smaller hysteresis than a harder implant (curve G).

It is noted that the dynamic behaviour of the embodiments of the inventive implant examined did not change over repeated compression cycles. In fact, the inventive implants have been subjected to repeated compression and decompression cycles ranging up to 1,000 cycles without any noticeable deterioration of their dynamic behaviour. In view of the chosen height of 3 cm for the conducted experiments and the predictable and stable dynamic behaviour up to deformations past 30% as the deformation-force curves did not reach their global maxima up to that point, it may be assumed that the examined embodiments of the inventive implants can be safely used in a dynamization range of 0-8 mm, for example. This is not the case for the examined ordinary implants, as their corresponding deformation-force curves (see curves in diagram of FIG. 5B) reach their global maxima already at or close to 5% deformation.

FIG. 6 illustrates the use of an implant 20 according to various embodiments for bone reconstruction or bone replacement therapy. The implant 20 according to various embodiments may be manufactured such that its top and bottom have contours that at least approximately match the shape of cut-off surfaces at a first bone part 61 and a second bone part 62, respectively. The cut-off surfaces at the first and second bone parts 61, 62 may be due to surgical bone removal. Once the implant 20 filled with the substance has been inserted between the cut-off surfaces of the first and second bone parts 61, 62, a load may be applied thereto by natural use (movement) of the corresponding body portion by the subject. This situation is similar to the experimental setup shown in FIGS. 3A, 3B and 4A, 4B where the implant 20 is arranged between two plates 31, 32 and compressed. Due to the flexibility and compressibility of the implant 20, the load is transferred to the substance inside the implant 20. It has been found that a rather flexible bone implant for containing bone graft material, in which the bone graft material is subject to reversible compression, is conducive to formation of solid and healthy bone matter, as it mimics a natural environment of a healing bone site, which is also subject to compression forces.

A further advantageous effect linked to the construction of the implants 20 according to various embodiments is the easy size trimming of the inventive implants 20 on site, i.e. during a surgery. Namely, when the implant 20 is provided in a limited number of sizes or just one size with regard to its height, i.e. its dimension along its main axis, its size may be adapted by the surgeon by removing the lowermost or uppermost circumferential line segment 11 together with the struts attached thereto. Expressed differently, the implant 20 may be trimmed by removing at least one circumferential layer 15. In the case of the implant 20 shown in FIG. 6, removing the lowermost or uppermost of the six circumferential layers 15 will lead to a fully operational implant 20 comprising five circumferential layers 15. Therefore, the inventive configuration of the implant 20 allows adjusting its size with a resolution of one circumferential layer 15 (which may practically have any desired dimension) without altering the dynamic properties of the implant 20.

In FIG. 7A a further embodiment of the inventive implant 70 is shown, namely one with a C-shaped body. At the same time, the implant 70 corresponds to one circumferential layer which comprises two circumferential line segments 11 with interconnecting struts 12, which are all slanted in one direction and attached to the circumferential line segments 11 at anchor points 13. The C-shaped implant 70 shares the common feature with the embodiments described so far in that it comprises a mesh formed into a continuous sidewall. A continuous sidewall may be also described as the circumferential line segment 11 forming a closed loop, which, as shown in FIG. 7A, does not necessarily have to be of round or circular shape. The C-shape of the implant 70 is characterized by an inward bulge or indentation 71, which may function as an accommodation for a bone nail. Due to the elasticity of the implant 70, it may be pressed towards a bone nail such that the inward bulge 71 snaps to and ultimately surrounds the bone nail. In particular, the channel-like entrance of the inward bulge 71 may have a width which is smaller than the diameter of the bone nail, which enables the snapping of the implant 70 around the bone nail.

FIG. 7B shows an implant 70 with multiple circumferential layers based on the C-shaped single circumferential layer implant 70 shown in FIG. 7A. The inward bulge 71 corresponds to an axial channel which is aligned along the main axis 21 of the implant 70.

As already noted above, the implant 20 according to various embodiments is able to reversibly deform under load by conversion of forces. As illustrated in FIG. 8, an axial load, represented by a downward compression force 81 being applied to the top of the implant 20 (corresponds to the scenario of FIGS. 3A, 3B and 4A, 4B) leads to compression of the entire implant 20. It goes without saying that the compression force may be also applied by an upward compression force being applied to the bottom of the implant 20 without affecting the underlying mechanical principle of operation of the implant. The compression of the implant 20 is achieved by rotation of the circumferential line segments 11 relative to one another. The rotation is induced by the struts 12 acting as force conductors from one circumferential line segment 11 to another. Since the struts 12 within one circumferential layer 15 are all slanted or biased in the same direction, this bias leads to a uniform transfer of force in one direction between the circumferential line segments 11. Therefore, the compression force acting on the implant 20 is stored therein by conversion thereof into internal rotation of the circumferential line segments 11 against each other which corresponds to torsion within the circumferential layers 15.

What is claimed is:

1. An implant for containing a substance, comprising a mesh formed into a continuous sidewall;
wherein the mesh comprises a plurality of circumferential line segments which are arranged above one another in the direction of a main axis of the implant and which are interconnected to one another by struts;
wherein the struts between two adjacent circumferential line segments are inclined in the same direction and wherein the inclination direction of struts arranged above a circumferential line segment is opposite to the inclination direction of struts arranged below that circumferential line segment.

2. Implant of claim 1, wherein the circumferential line segments and the struts comprise an elastic material.

3. Implant of claim 1, wherein the struts between two adjacent circumferential line segments are inclined at the same inclination angle.

4. Implant of claim 1, wherein the inclination angle of struts arranged above a respective circumferential line segment is the same as the inclination angle of struts arranged below that circumferential line segment.

5. Implant of claim 1, wherein the inclination angle of struts arranged above a respective circumferential line segment is different from the inclination angle of struts arranged below that circumferential line segment.

6. Implant of claim 3, wherein the inclination angle lies between 30° and 80°.

7. Implant of claim 1, wherein the number and/or a size of the of struts arranged above a respective circumferential line segment is the same as the number and/or the size of the of struts arranged below that circumferential line segment.

8. Implant of claim 1, wherein the number and/or a size of the of struts arranged above a circumferential line segment is different from the number and/or the size of the of struts arranged below that circumferential line segment.

9. Implant of claim 1, wherein at least one anchor point of a strut connecting to a circumferential line segment from below is aligned with an anchor point of a strut connecting to that circumferential line segment from above.

10. Implant of claim 1, wherein anchor points of struts connecting to a circumferential line segment from below are shifted relative to the anchor points of struts connecting to that circumferential line segment from above.

11. Implant of claim 1, wherein the implant has a Poisson's ratio of approximately zero.

12. Implant of claim 1, wherein a bottom circumferential line segment and/or a top circumferential line segment each define a contour which matches a contour of a corresponding portion of a bone which is to be contacted by the bottom and the top of the implant.

13. Implant of claim 1, wherein each of the circumferential line segments defines a plane which is arranged perpendicularly to the main axis of the implant.

14. Method for manufacturing an implant for containing a substance, the method comprising:
forming a plurality of circumferential line segments interconnected by struts to obtain a mesh formed into a sidewall of the implant;
wherein the mesh comprises a plurality of circumferential line segments which are arranged above one another in the direction of a main axis of the implant and which are interconnected to one another by struts;
wherein the struts between two adjacent circumferential line segments are inclined in the same direction and wherein the inclination direction of struts arranged above a circumferential line segment is opposite to the inclination direction of struts arranged below that circumferential line segment.

15. Method for adjusting the size of an implant as defined in claim 1 for containing a substance, the method comprising:
removing at least one circumferential line segment and the struts attached thereto to obtain an implant with a reduced axial dimension.

16. The implant of claim 6, wherein the inclination angle lies between 40° and 70°.

17. The implant of claim 1, wherein the implant is a bone implant.

18. The implant of claim 17, wherein the substance contained in the implant is a graft material.

19. The implant of claim 18, wherein the graft material comprises biological cell material or synthetic bone graft material.

20. A method of bone reconstruction or bone augmentation surgery, wherein the method comprises implanting into the body of subject an implant that contains a substance,
wherein the implant comprises a mesh formed into a continuous sidewall;
wherein the mesh comprises a plurality of circumferential line segments, which are arranged above one another in the direction of a main axis of the implant and which are interconnected to one another by struts;
wherein the struts between two adjacent circumferential line segments are inclined in the same direction and wherein the inclination direction of struts arranged above a circumferential line segment is opposite to the inclination direction of struts arranged below that circumferential line segment.
